(19) United States
(12) Patent Application Publication
(10) Pub. No.: US 2010/0324303 A1
(43) Pub. Date: Dec. 23, 2010

(54) Use of Tetramic Acid Derivatives for Controlling Animal Pests After Treatment of the Trunk, the Branches, the Influorescences or the Buds

(75) Inventor: Reiner FISCHER, Monheim (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/867,732

(22) PCT Filed: Feb. 6, 2009
(86) PCT No.: PCT/EP09/00816

§ 371 Date: Aug. 13, 2010

(30) Foreign Application Priority Data
Feb. 13, 2008 (EP) 08151372.3

(51) Int. Cl. C07D 209/54

(52) U.S. Cl. 548/408

(57) ABSTRACT

The present invention relates to the use of tetramic acid derivatives of the formulae (I) or (II)

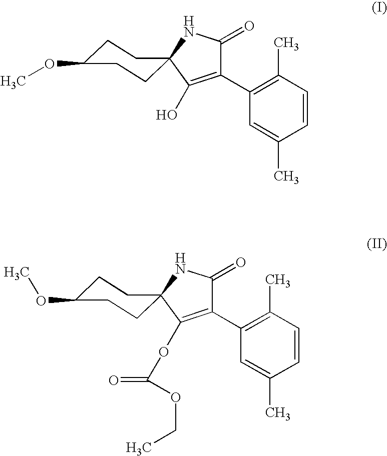

for controlling animal pests after stem treatments (spraying on, painting on, injecting, applying), treatments of branches, stalks, shoots and twigs (painting on, spraying on, applying), influorescence treatments and bud treatments by injection.

The present invention relates to the use of tetramic acid derivatives for controlling animal pests after stem treatments (spraying on, painting on, injecting, applying), treatments of branches, stalks, shoots and twigs (painting on, spraying on, applying), influorescence treatments and bud treatments by injection.

Tetramic acid derivatives (WO 98/05638) and their cis isomers (WO 04/007448) which have insecticidal and/or acaricidal activity are known.

The use of tetramic acid derivatives against spider mites and insects after pouring on, trickle application or soil injection (WO 07/126691) and against nematodes after foliar application (U.S. application. No. 61/008,507), but also the control of plant diseases following drench application (EP application number 07150293), are furthermore known.

Other uses are described in WO 2007/131681.

Surprisingly, it has now been found that the compounds of the formulae (I) or (II)

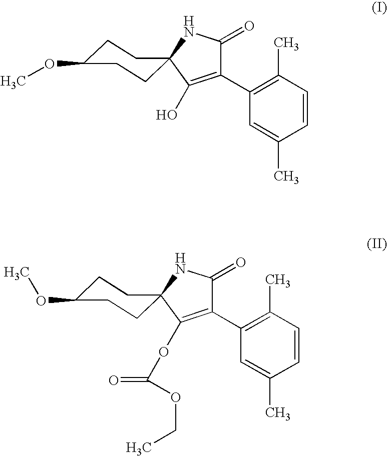

are also well suited for controlling insects and spider mites after stem treatment (for example by painting on, spraying on, applying, injecting), after the treatment of stalks, branches, twigs and shoots (for example by painting on, spraying on, applying) and after injection into flower buds and influorescences (referred to as bud injection in expert circles).

Accordingly, the present invention relates to the use of tetramic acid derivatives for controlling insects and spider mites after stem applications, branch applications, stalk applications, twig applications and shoot applications and also influorescence and flower bud treatments in the open or in enclosed systems (for example greenhouses or under foil cover) in annual crops (for example ornamentals) and in perennial crops (for example tropical fruit, citrus, conifers, pome fruit, stone fruit, hops).

The crops to be protected, which are only described in general terms, are hereinbelow differentiated and specified in a more detailed manner.

Thus, the use in perennial crops is understood as meaning citrus such as, for example, oranges, grapefruits, tangerines, lemons, limes, Seville oranges, kumquats, satsumas;

but also pome fruit such as, for example, apples, pears and quinces, and stone fruit such as, for example, peaches, nectarines, cherries, plums, quetsch, apricots;
furthermore grapevines, hops, olives, tea and tropical crops such as, for example, mangoes, papayas, figs, pineapples, dates, bananas, durians, kaki fruit, coconuts, cacao, coffee, avocados, lychees, maracujas, guavas, oil palms;
furthermore almonds and nuts such as, for example, hazelnuts, walnuts, pistachios, cashew nuts, Brazil-nuts, pecan nuts, butternuts, chestnuts, hickory nuts, macadamia nuts, peanuts,
in addition also soft fruits such as, for example, currants, gooseberries, raspberries, blackberries, blueberries, strawberries, cranberries, kiwi fruit, American cranberries.

As regards the use, ornamentals are understood as meaning annual and perennial plants, for example cut flowers such as, for example, roses, carnations, gerbera, lilies, margarites, chrysanthemums, tulips, narcissus, anemones, poppies, amaryllis, dahlias, azaleas, hibiscus,

but also for example border plants, pot plants and perennials such as, for example, roses, Tagetes, violas, geraniums, fuchsias, hibiscus, chrysanthemums, busy Lizzie, cyclamen, African violet, sunflowers, begonias,
furthermore for example shrubs and conifers such as, for example, ficus, rhododendrons, firs, spruces, pines, yews, umbrella pines, oleander.

Preferred among the family Thripidae are: Frankliniella spp. Thrips spp., Heliothrips spp., Hercinothrips spp., Caliothrips spp., Scirthothrips spp. in crops such as, for example, soft fruit, ornamentals, potatoes, tropical crops, grapevines, nuts, citrus, tea.

Preferred from the family Pseudococcidae are: Pericerga, Pseudococcus spp., Planococcus spp., Dysmicoccus spp., in crops such as, for example, citrus, stone fruit and pome fruit, tea, grapevines, ornamentals and tropical crops.

Preferred from the family Coccidae are: Ceroplastes spp., Drosicha spp., Pulvinaria spp., Protopulminaria spp., Saissetia spp., Coccus spp., in perennial crops such as, for example, citrus, pome fruit, stone fruit, olives, grapevines, coffee, tea, tropical crops, ornamentals.

Preferred from the family Diaspididae are: Quadraspidiotus spp., Aonidiella spp., Lepidosaphes spp., Aspidiotus spp., Parlatoria spp., Pseudaulacaspis spp., Unaspis spp., Pinnaspis spp., Selenaspidus spp., in crops such as, for example, citrus, pome fruit, stone fruit, almonds, nuts, olives, tea, ornamentals, grapevines, tropical crops.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or else by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, thrips and seeds.

The treatment according to the invention of the plants and plant parts with the active substance is effected directly or by acting on their environment, habitat or store by the customary treatment methods, for example by dipping, spraying, vaporizing, atomizing, scattering, painting on, injecting and, in the case of propagation material, especially in the case of seeds, furthermore by coating with one or more coats.

As already mentioned above, all plants and their parts can be treated in accordance with the invention. In a preferred embodiment, plant species and plant varieties which are found in the wild or which are obtained by traditional biological breeding methods, such as hybridization or protoplast fusion, and parts of these species and varieties are treated. In a further preferred embodiment, transgenic plants and plant varieties which have been obtained by recombinant methods, if appropriate in combination with traditional methods (genetically modified organisms) and their parts are treated. The term “parts”, “parts of plants” or “plant parts” have been described above.

Plants which are especially preferably treated in accordance with the invention are those of the plant varieties which are in each case commercially available or in use. Plant varieties are understood as meaning plants with novel traits which have been bred both by conventional breeding, by mutagenesis or by recombinant DNA techniques. They may take the form of varieties, biotypes or genotypes.

Depending on the plant species or plant varieties, their location and growth conditions (soils, climate, vegetation period, nutrition), superadditive (“synergistic”) effects may also occur as a result of the treatment according to the invention. Effects which exceed the effects actually to be expected are, for example, reduced application rates and/or widened activity spectrum and/or an enhancement of the activity of the substances and compositions which can be used in accordance with the invention, better plant growth, increased tolerance to high or low temperatures, increased tolerance to drought or to water or soil salinity, increased flowering performance, facilitated harvest, speedier maturation, higher yields, higher quality and/or higher nutritional value of the crop products, better storability and/or processability of the crop products.

The preferred transgenic plants or plant varieties (plants or plant varieties obtained by means of genetic engineering) which are to be treated in accordance with the invention include all plants which, by means of the recombinant modification, have received genetic material which confers particularly advantageous valuable traits to these plants. Examples of such traits are better plant growth, increased tolerance to high or low temperatures, increased tolerance to drought or to water or soil salinity, increased flowering performance, facilitated harvest, speedier maturation, higher yields, higher quality and/or higher nutritional value of the crop products, better storability and/or processability of the crop products. Other examples of such traits which are particularly emphasized are an improved defence of the plants against animal and microbial pests such as insects, mites, phytopathogenic fungi, bacteria and/or viruses, and an increased tolerance of the plants to specific herbicidally active compounds. Examples of transgenic plants which are particularly emphasized are the important crop plants such as cereals (wheat, rice), maize, soybean, potato, cotton, tobacco, oilseed rape and fruiting plants (with the fruits apples, pears, citrus fruits and grapes), with particular emphasis on maize, soybean, potato, cotton, tobacco and oilseed rape. Traits which are particularly emphasized are the increased defence of the plants against insects, arachnids, nematodes and slugs and snails as a result of toxins formed in the plants, in particular toxins which are produced in the plants by the genetic material of Bacillus thuringiensis (for example by the genes CryIA(a), CryIA(b), CryIA(c), CryIIA, CryIIIA, CryIIIB2, Cry9c, Cry2Ab, Cry3Bb, and CryIF and their combinations) (hereinbelow “Bt plants”). Traits which are also particularly emphasized are the increased defence of plants against fungi, bacteria and viruses by systemic acquired resistance (SAR), systemin, phytoalexins, elicitors and resistance genes and correspondingly expressed proteins and toxins. Traits which are furthermore especially emphasized are the increased tolerance of the plants to specific herbicidally active compounds, for example imidazolinones, sulfonylureas, glyphosate or phosphinothricin (for example “PAT” gene). The specific genes which confer the desired traits can also occur in combinations with one another in the transgenic plants. Examples of “Bt plants” which may be mentioned are maize varieties, cotton varieties, soybean varieties and potato varieties which are sold under the trade names YIELD GARD® (for example maize, cotton, soybean), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soybean varieties which are sold under the trade names Roundup Ready® (glyphosate tolerance, for example maize, cotton, soybean), Liberty Link® (phosphinothricin tolerance, for example oilseed rape), IMI® (imidazolinone tolerance) and STS® (sulfonylurea tolerance, for example maize). Herbicide-resistant plants (bred conventionally for herbicide tolerance) which may also be mentioned are the varieties sold under the name Clearfield® (for example maize). Naturally, what has been said also applies to plant varieties which will be developed, or marketed, in the future and which have these genetic traits or traits to be developed in the future.

The active compounds of the formulae (I) and (II) can be converted into the customary formulations, such as solutions, emulsions, wettable powders, suspensions, powders, dusts, pastes, soluble powders, granules, suspoemulsion concentrates, natural and synthetic materials impregnated with active compound, and ultrafine encapsulations in polymeric materials.

These formulations are produced in the known manner, for example by mixing the active compound with extenders, that is, liquid solvents and/or solid carriers, optionally with the use of surfactants, that is, emulsifiers and/or dispersants and/or foam formers.

In the case of the use of water as an extender, organic solvents can, for example, also be used as cosolvents. Liquid solvents which are suitable are mainly: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example mineral oil fractions, mineral oils and vegetable oils, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulfoxide, and water.

Solid carriers which are suitable are:

for example ammonium salts and ground natural minerals, such as kaolins, clays, talc, chalk, quartz, attapulgite, montmorillonite or diatomaceous earth, and ground synthetic minerals, such as highly-disperse silica, alumina and silicates; suitable solid carriers for granules are: for example crushed and fractionated natural rocks such as calcite, marble, pumice, sepiolite and dolomite, and synthetic granules of inorganic and organic meals, and granules of organic material such as sawdust, coconut shells, maize cobs and tobacco stalks; suitable emulsifiers and/or foam formers are: for example non-ionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates as well as protein hydrolysates; suitable dispersants are: for example lignin-sulfite waste liquors and methylcellulose.

Adhesives such as carboxymethylcellulose and natural and synthetic polymers in the form of powders, granules or latices, such as gum arabic, polyvinyl alcohol and polyvinyl acetate, and natural phospholipids, such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general comprise between 0.1 and 95% by weight of active compound, preferably between 0.5 and 90%, and additionally preferably extenders and/or surfactants.

The active compound content of the use forms prepared from the commercially available formulations can vary within wide ranges. The active compound concentration of the use forms can be in the range of from 0.0000001 up to 95% by weight of active compound, preferably between 0.0001 and 1% by weight.

Application is in a customary manner which is appropriate for the use forms.

### USE EXAMPLES

### Thrips

### Thripidae

Very especially preferred is the control of the following species from the family thrips (Thripidae) in the following crops following influorescence and bud treatment:

| | |
|---|---|
| Frankliniella occidentalis | in soft fruit, in ornamentals such as roses, hibiscus, |
| Frankliniella schultzei | chrysanthemums and in potatoes and tropical crops such as, for |
| Frankliniella fusca | example, papayas, avocado, conifers |
| Thrips palmi | in soft fruit, in ornamentals such as, for example, roses, hibiscus, in |
| Thrips tabaci | tropical crops such as, for example, papayas, pineapples, bananas, |
| Thrips hawaiiensis | grapevines, nuts |
| Heliothrips | in ornamentals such as, for example, roses, hibiscus, azaleas, |
| haemorrhoidalis | tropical crops such as guavas, citrus such as, for example, lemons, |
| | oranges, grapevines, nuts such as, for example, macadamia nuts |
| Hercinothrips femoralis | in tropical crops such as, for example, bananas, ornamentals |
| Hercinothrips bicinctus | |
| Hercinothrips phaseoli | |
| Caliothrips phaseoli | in tropical fruit such as, for example, avocados |
| Scirthothrips aurantii | in citrus such as, for example, oranges, lemons, grapefruits, |
| Scirthothrips dorsalis | tangerines, ornamentals, tea |
| Scirthothrips citri | |

### Pseudococcidae

Very especially preferred is the control of the following species from the family Pseudococcidae in the following crop following stem application:

| | |
|---|---|
| Pseudococcus citri | in citrus such as, for example, oranges, tangerines, grapefruits, |
| Pseudococcus comstocki | limes, lemons, satsumas, in pome fruit such as, for example, apples |
| Pseudococcus maritimus | and pears, in grapevines, in ornamentals, in tropical crops such as, |
| | for example, pineapples, bananas |
| Dysmicoccus boninsis | in pome fruit such as, for example, apples, pears, in tea, in tropical |
| Dysmicoccus cryptus | crops such as, for example, pineapples, guyabano, bananas |
| Dysmicoccus brevipes | |
| Planococcus lilacinus | in citrus such as, for example, oranges, tangerines, grapefruits, |
| Planococcus citri | limes, lemons, satsumas, in grapevines |
| Pericerga purchasi | in citrus such as, for example, oranges, tangerines, grapefruits, |
| | limes, lemons, satsumas |

### Coccidae

Very especially preferred is the control of the following species from the family Coccidae in the following crops, preferably after stem application:

| | |
|---|---|
| Ceroplastes ceriferus | in citrus such as, for example, oranges, grapefruits, tangerines, |
| Ceroplastes floridensis | lemons, limes, satsumas |
| Ceroplastes rubens | |
| Ceroplastes rusci | |
| Drosicha mangiferae | in tropical crops, for example mangoes |
| Drosicha stebbengii | |
| Pulvinaria aurantii | in citrus such as, for example, oranges, grapefruits, tangerines, |
| Pulvinaria aethiopicus | lemons, limes, satsumas, in grapevines |
| Pulvinaria vitis | |
| Protopulminaria pyriformis | in pome and stone fruit |
| Saissetia oleae | in citrus such as, for example, oranges, grapefruits, tangerines, |
| Saissetia nigra | limes, |
| | in citrus such as, for example, lemons, satsumas, in olives, in |
| | tropical crops, for example bananas |
| Coccus viridis | in citrus such as, for example, oranges, tangerines, grapefruits, |
| | limes, lemons, satsumas, in tropical crops, for example pineapples |
| Coccus hesperdium | in pome fruit such as, for example, apples, pears, in stone fruit such |
| | as, for example, peaches, nectarines, plums, apricots, cherries, in |
| | coffee, in olives, in tea, in grapevines |

### Diaspididae

Very especially preferred is the control of the following species from the family Diaspididae in the following crops, preferably after stem application:

| | |
|---|---|
| Quadraspidiotus | in citrus such as, for example, oranges, tangerines, Citrus limonum, |
| perniciosus | grapefruits, in pome fruit such as, for example, apples, pears, quinces, in |
| Quadraspidiotus | stone fruit such as, for example, peaches, nectarines, apricots, plums, |
| juglansregiae | cherries, in nuts such as, for example, almonds, pistacchios, walnuts, |
| | hazelnuts, in ornamentals such as, for example, shrubs, conifers, pot |
| | plants, in tropical crops, for example lychees |
| Aonidiella aurantii | in citrus such as, for example, oranges, tangerines, grapefruits, limes, |
| Aonidiella citrina | lemons, satsumas |
| Lepidosaphes ulmi | in citrus such as, for example, oranges, tangerines, grapefruits, limes, |
| Lepidosaphes beckii | lemons, satsumas, in pome fruit such as, for example, apples and pears, |
| | in stone fruit such as, for example, peaches, nectarines, plums, apricots, |
| | cherries |
| Aspidiotus destructor | in citrus such as, for example, oranges, tangerines, grapefruits, limes, |
| Aspidiotus hederae | lemons, satsumas, in ornamentals such as, for example, shrubs, pot |
| Aspidiotus nerii | plants, in olives, in tropical crops, for example mangoes, Citrus limonum |
| Aspidiotus ficus | |
| Pseudaulacaspis | in pome fruit such as, for example, apples, pears, in stone fruit such as, |
| pentagona | for example, peaches, apricots, nectarines, cherries, plums, quetsch, in |
| | tea |
| Unaspis yanonensis | in citrus such as, for example, oranges, tangerines, limes, grapefruits, |
| Unaspis citri | lemons, satsumas, in tropical crops, for example, pineapples, mangoes |
| Pinnaspis aspidistrae | in citrus such as, for example, oranges, tangerines, limes, lemons, |
| Parlatoria ziziphus | satsumas, grapefruits, in olives |
| Parlatoria pergandei | |
| Parlatoria oleae | |
| Selenaspidus | in citrus such as, for example, oranges, tangerines, limes, grapefruits, |
| articulatus | lemons, satsumas |

### Example 1

Banana trees cv. “Cavendish” are treated in three replications against banana flower thrips (Thrips hawaiiensis). Here, the active substances Example (II) (100 OD) are tested versus the commercial standard imidacloprid (100 SL) at the application rates specified. The application is carried out by injecting in each case 80 ml of injection solution into the flower buds. In each case three flower buds are treated. The evaluation is effected 3 and 91 days after the treatment by scoring the destruction of the population on the influorescences and bunches.

| | | |
|---|---|---|
| | | Activity in |
| Active | Application | % Abbott |

| substance | rate (g) a.i./l | 3 d | 91 d |
|---|---|---|---|
| Imidacloprid | 0.1 | 98.4 | 100 |
| Example (II) | 0.3 | 66.7 | 100 |

### Example 2

Banana trees (growth stage BBCH 1130) cv. “Cavendish” consisting of mother plant and sucker are treated in three replications following foliar treatment against Aspidiotus destructor and against the pineapple mealybug Dysmicoccus brevipes by pseudostem application. To this end, in each case 100 ml of spray solution are applied, in each case 50 ml being sprayed onto the leaves and 50 ml onto the stem. Before the stem application, the outermost stem layer is first peeled off, which is general practice. Here, the active substance (II) (SC 240) is tested in a tank mix along with 0.025% Hoestick (XL 500) versus the commercial standards chlorpyrifos-methyl (500 EC) and liquid paraffin (Banole oil) (EC 600).

The evaluation is carried out 3, 7, 14 and 22 days after the treatment by scoring the destruction of the pineapple mealybug on the stem.

| Active | | Activity (%) Abbott | | | |
|---|---|---|---|---|---|
| substance | Application rate g/l | 3 d | 7 d | 14 d | 22 d |
| Banole oil | 30 | 87.9 | 91.7 | 89.0 | 96.5 |
| Chlorpyrifos- | 0.8 | 98.9 | 99.3 | 100.0 | 100.0 |
| methyl | | | | | |
| Example (II) | 0.3 | 81.2 | 92.3 | 96.4 | 96.7 |

### Example 3

Young orange trees cv. “Valencia” which are approximately 5 years old are treated in three applications against the California red scale Aonidiella aurantii after stem application by painting on the active substances. Here, the active substance (II) (SC 240) is tested versus the commercial standard imidacloprid at the application rates detailed. The evaluation is carried out 205 days after the treatment by scoring the infestation on the fruits.

| Active | Application rate | Efficacy |
|---|---|---|
| substance | g a.i./tree | (% Abbott) |
| Imidacloprid | 2 | 100 |
| Example (II) | 4 | 100 |

We claim:

1. The use of compounds of the formulae (I) or (II) 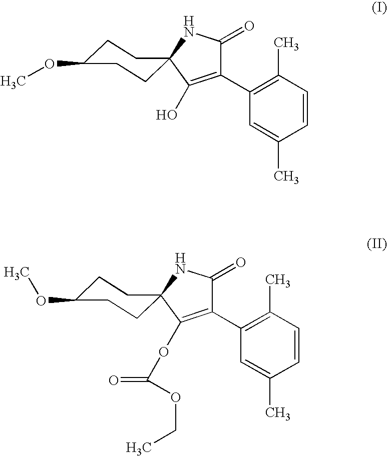
for controlling insects and spider mites after stem treatment, after the treatment of stalks, branches, twigs and shoots and after injection into flower buds and influorescences.

2. The use of compounds of the formulae (I) or (II) as claimed in claim 1 for controlling thrips after influorescence treatment.

3. The use of compounds of the formulae (I) or (II) as claimed in claim 1 for controlling thrips after bud treatment.

4. The use of compounds of the formulae (I) or (II) as claimed in claim 1 for controlling Pseudococcidae after stem treatment.

5. The use of compounds of the formulae (I) or (II) as claimed in claim 1 for controlling Cococcidae after stem treatment.

6. The use of compounds of the formulae (I) or (II) as claimed in claim 1 for controlling Diaspididae after stem treatment.

7. The use of the compound of the formula (I) as claimed in claims 1-6.

8. The use of the compound of the formula (I) as claimed in claims 1-6.
